(19) United States
(12) Patent Application Publication
(10) Pub. No.: US 2022/0007938 A1
(43) Pub. Date: Jan. 13, 2022

(54) AN AUTOMATED EXAMINATION SYSTEM AND METHOD

(71) Applicant: MIKAJAKI, SA, Plan Les Ouates (Geneva) (CH)

(72) Inventors: Michael ASSOULINE, Paris (FR); Joel HOCHNER, Modiin (IL); John SPEYRER, Le Grand Saconnex, Geneva (CH)

(73) Assignee: MIKAJAKI, SA, Plan Les Ouates (Geneva) (CH)

(21) Appl. No.: 17/295,793

(22) PCT Filed: Nov. 21, 2019
(86) PCT No.: PCT/IL2019/051270

§ 371 Date: May 20, 2021

(30) Foreign Application Priority Data
Nov. 22, 2018 (IL) 263233

(51) Int. Cl. A61B 3/18; A61B 3/00; A61B 3/12; A61B 5/00

(52) U.S. Cl. CPC A61B 3/18; A61B 3/0083; A61B 5/441; A61B 5/0088; A61B 3/12

(57) ABSTRACT

Provided is a system for automatic examination of a body part of a subject, including a support configured to support a subject's body portion including said body part; a measuring subsystem including at least one sensor configured to detect at least one feature of the subject, a mechanical subsystem configured for receiving at least two examination devices and for positioning the at least two examination devices and/or said support in operative positions; and a control unit.

### TECHNOLOGICAL FIELD

The present disclosure relates to automatically operated examination systems and specifically to automated systems for ophthalmological examination and a method of automatic ophthalmological examination.

### BACKGROUND ART

References considered to be relevant as background to the presently disclosed subject matter are listed below:

U.S. Pat. No. 5, 841,502

US patent application No. 2009/0182311

International patent application publication No. WO2016/022347

Japanese patent application No. 2004089216

US patent application No. 2013/0286353

US patent application No. 2018/0078134.

Acknowledgement of the above references herein is not to be inferred as meaning that these are in any way relevant to the patentability of the presently disclosed subject matter.

### BACKGROUND

Automated systems for medical examination have been previously reported. For example, U.S. Pat. No. 5,841,502 describes a system whereby an ophthalmological apparatus adjusts its alignment to be aligned with an eye of a subject to be examined. US2009/0182311 relates to a system for contacting an eye to secure the eye in position. WO2016/022347 describes a medical robotic work station. JP2004089216 describes an ophthalmological apparatus capable of being operated by a user to keep a forehead pad part clean even if a patient differs. US2013/0286353 describes an arrangement for the automatic rough positioning of an individual in front of a particular piece of ophthalmological equipment. US2018/0078134 describes an eye examination kiosk and method.

### GENERAL DESCRIPTION

Various types of examination systems and methods are used to diagnose and monitor different regions of a subject's body. The success of such examinations often depends on the accurate positioning of the subject, or at least the region of the subject to be examined, with respect to the examination devices. Optimization of the positioning, as well as the stability of the subject during examination (ensuring little to no movement of the subject during examination), may result in an accurate and meaningful diagnosis.

For example, in examination of tissues or portions of a subject's face, e.g situations of ophthalmological and dental examinations, 3D imaging or dental CT scans, very accurate and stable positioning of the subject and of the examination devices with respect to the subject is required. Such stability and precise positioning of the subject allows accurate results to be achieved when the scanning involves focusing and/or building up a large 2D or even a 3D image or model based on multiple scans which must be joined together.

In ophthalmology, examinations are used to diagnose ocular defects and conditions, and/or for routine diagnostics. Eye conditions may be conditions contributing to loss of quality of life or to uncontrolled progression of potentially visually impairing or blinding disease. Non-limiting examples of eye conditions include visual acuity, poor visual correction, dry eye syndrome, ocular manifestation of systemic disease, tumor, keratoconus, diabetic retinopathy, glaucoma, macular degeneration, retinal detachment and other ocular pathologies, During the ophthalmology examination, a subject to be examined is stably positioned to minimize movements that may affect the data acquisition and its accuracy. This positioning is usually achieved by an array of elements, such as a chin support and a forehead support for example, that can be manually adjusted either by the examined individual or by an operator, to support the head stably in place. This requires taking into account the shape and/or proportions of the face if a facial feature is to be examined for example, so that the face as a whole can be correctly positioned. Additionally, the examination machine or device itself needs to be aligned with the specific facial feature to be examined, for example, the alignment of the eye, pupil, iris and/or other part of the eye must be maintained in a stable position during the course of examination. in certain circumstances, very high accuracy of positioning is required, for example, in retina imaging systems which image the retina through the pupil.

Additionally, in ophthalmology, and indeed in any other examination, these supports, which are in contact with the individual, should be kept clean and sanitary to ensure adequate hygiene and prevent cross-contamination between examined individuals. In ophthalmology, the cleaning is currently achieved by manual wiping, spraying or changing disposable paper strips between successive examinations. In other fields, for example, with supports such as an examination bed, disposable paper is dispensed from a roll and replaced manually between examination of different patients.

The present disclosure is based on the development of an examination system that may be implemented in medical work stations or biometric identification stations such as, but not limited to, ophthalmological examination stations. The system described herein is capable of automatically adjusting the subject's position for the examination and automatically position at least two examination devices. This reduces the need for, and even eliminates, external operator intervention while providing reliable and accurate results, and hence may be considered as an autonomous examination system.

Specifically, and as described herein below, the examination system is configured to automatically and accurately position at least two examination devices may be the same or different). The examination devices may provide additive measured data (for example in case a low signal is obtained) or different measured data. The capability of the examination system described herein to automatically position two or more examination devices allows a comprehensive characterization of a subject's condition, for example an eye condition.

The ability to accurately position at least two different examination devices—which may be devices having different dimensions, focusing distance requirements and other constraints—is uniquely achieved in accordance with this disclosure by allowing such devices to change location within a relatively limited space so as to be interchangeably and automatically positioned in front of the subject's face in an examination relationship therewith; e.g. with an optical examination device having its optical axis aligned with that of the eye. Such developments require careful consideration of many factors including, but not limited to, space, positioning, storage, conveying, controlling, receiving output data, etc. from at least two (i.e. multiple) examination machines which may have different sources.

In addition, the system described herein may be adopted for simultaneous measurements of more than a single individual at the same time, permitting the examined individuals to undergo the same or a different type of examination simultaneously.

According to a first aspect of the present disclosure, there is provided a system for automatic examination of a body part of a subject, comprising a support, a measuring subsystem, a mechanical subsystem and a control unit.

The support being configured to support a body portion comprising* said body part so as to fix said body part in a fixed examination position;

the measuring subsystem comprising at least one sensor configured to detect a feature of the subject;

the mechanical subsystem configured for receiving at least two examination devices and for positioning the at least two devices and/or said support in operative positions (namely a position in which the device so positioned can perform its examination); and

the control unit configured for (i) receiving feature data from the measuring subsystem representative of the at least one feature and determining the operative positions based thereon, the operative positions being selected to permit the at least two examination devices to examine said body part, (ii) outputting instructions to said mechanical subsystem for said positioning of said support and/or the at least two examination devices in said operative positions, (iii) receiving examination data from the at least two examination devices representative of parameters of the body part examined thereby, and (iv) outputting the examination data, or one or more images representative thereof.

According to one embodiment of this disclosure (the “ophthalmology embodiment”), the examined body part is the eye, said support is a head support and said at least two devices are ophthalmological devices.

Other embodiments of this disclosure are systems intended, for example, for dental examination or dermatological examination and comprising, respectively, dental examination devices and dermatological examination devices.

In the description specific reference will be made to the ophthalmology embodiment. This specific reference has the purpose of illustrating this disclosure of all its aspects and is not intended as limiting in any way. The specifically described embodiment apply, maxis mutandis, to other embodiments of this disclosure.

The system described herein is an automatic system. The term “automatic” as used herein is meant to denote that the system may be operated without the need for human or operator intervention, or any external interference, in order to correctly suit, or conform, the head support and/or the at least two ophthalmological examination devices to the particular subject to be examined and/or to align the portion of the subject to be examined with each of the at least two ophthalmological examination devices. Specifically, the positioning of the at least two ophthalmological. examination devices and/or the head support is carried out in response to the feature data as described herein. This arrangement is thus fully automated, i.e. automatically self-adjusting (also denoted as autonomous system).

The head support in accordance with the present disclosure may be located within a subject examination area that is an area in which a subject to be examined is located when examination is to take place. This examination area may be a dedicated standing place, seat, position on the ground where a chair or wheelchair may be placed, a cubicle, set area or the like. Since the head support is located in a subject examination area and not attached to a particular examination device, the head support is suitable for use in conjunction with any examination device, and there is no need to provide, and adjust, multiple head supports. Thus a single subject examination area comprising a single head support may be suitable for use with a variety of examination devices as described herein. The head support may support any portion of the head. In some embodiments, the head portion is a chin. In some other embodiments, the head portion is a forehead. In some embodiments, the head support is configured to support the chin, the forehead or a combination thereof. In some other embodiments, the head support is a chin rest support, a forehead support or a combination thereof. Thus the head support can be configured to support a chin and/or a forehead.

Measurement of the subject is done by at least one sensor in conjunction with the measuring subsystem. The sensor may be any one or more sensors capable of sensing data about a subject to be examined and in accordance with the present disclosure, is configured to detect at least one feature of an examined subject. The at least one feature, such as a chin or forehead of a subject, can be measured within a frame of reference as having specific X, Y and Z coordinates, measured for example relative to one or more reference surfaces and/or points. The reference surfaces and/or points tray be, for example situated on the floor of the examination area, or may comprise any predetermined surface of the examination area or any piece of furniture present in the examination area, or a specific point or points on any one or more of these. Thus the control system tray be configured to determine a position of a chin or a forehead of the subject relative to the at least one reference surface and/or point, for example in an X, Y, Z coordinate system. The reference point or surface may be within the system, for example a bottom surface of the system or the head support. Alternatively or additionally, the controller in conjunction with the measurement subsystem may be configured to determine a relative position of the eyes, chin or other feature of a subject relative to a. point or surface in the examination area such as a head support or other moving or fixed apparatus, so as to allow the head support to be conformed and correctly positioned relative to the particular subject.

The sensor may, for example, comprise one or more of a camera, infra-red sensor, pressure sensor, temperature sensor, proximity sensor and light sensor. In some embodiments, the sensor may comprise at least one imaging system. In some embodiments, the imaging system may comprise at least one camera or video camera. The measuring subsystem may comprise an analysis module, configured to analyze data from the sensor, e.g. images detected by the imaging system, and output measurement or feature data.

In some embodiments, the sensor comprises at least one motion detector. A motion detector is a device capable of detecting movement such as a moving subject to be examined. A motion detector may be capable of detecting the position of a subject, and accordingly indicating the presence or absence of an individual in proximity to the apparatus. Possible motion detectors which may be used include, by way of non-limiting example only, passive infrared (PIR) sensors, ultrasonic transducers, cameras and 3D scanners.

The feature data from the measuring subsystem may comprise any anthropometric data, encompassing proportions of the subject to be examined (for example, in dental or ophthalmological examination systems, useful anthropometric data may include height when seated or when standing, size of head, position of eyes in the head, i.e, distance from chin and forehead, spacing between the eyes, etc.). In some embodiments, the feature is anthropometric data including a geometrical mapping of various body regions, such as, but not limited to one or more of a knee, hip, shoulder, chin and eye.

In some embodiments, the feature is a facial feature. In some other embodiments, the facial feature, is selected from the group consisting of chin, forehead, eyes and nose. The at least one facial feature can be one or both eyes. In an exemplary ophthalmological examination system, the sensor for example a camera) may be capable of obtaining an image of at least one eye, and may be capable of tracking movement of the eye and/or a feature of the eye, for example the iris, pupil, etc. The eye or pupil movement as used herein encompasses eye blinking, frequency of blinking and pupil movement.

The feature data can correspond to at least one of the distances eye to eye, pupil to pupil, eye to nose, eye to forehead, eye to chin, chin to forehead, nose to forehead and chin to nose.

In accordance with some embodiments, the sensor may sense the presence of a subject in the examination area and measure at least one feature datum of the subject prior to examination.

The mechanical subsystem, which is part of the system described herein, is configured to receive at least two examination devices. Although in the present disclosure, the at least two examination devices are ophthalmological examination devices, it is envisaged that such a system could alternatively or additionally be suitable for use with at least one of dental examination devices or dermatological examination devices. In some embodiments, the at least two examination devices are different ophthalmological examination devices. In some other embodiments, the at least two examination devices are identical ophthalmological examination devices. In some embodiments, the mechanical subsystem is configured to receive more than two ophthalmological examination devices.

For the purpose of the examination system, the at least two ophthalmological examination devices, as well as the head support, should be in a proper mutual orientation, both with respect to each other and to the examined subject. The proper orientation is denoted herein as the “operative position”, referring to a functioning or effective position. The mechanical subsystem is configured to position each of the at least two examination devices and/or the head support in the operative position. Positioning in the operative position can be based. on alignment of an optical axis of an examination device with an optical axis of an eye of a subject.

The mechanical subsystem may comprise an adjustment means for positioning the head support and/or the at least two examination devices in an appropriate position, namely the operative position. The mechanical subsystem may comprise, for example lead screws and/or rack and pinion. The mechanical subsystem may also comprise a robotic part such as a robotic arm allowing rotational motion or translation displacement. For example, the dimensions, curvature and position of the head support may be adjusted to fit the particular shape of the subject to be examined. This is necessary because a child will have smaller dimensions and different proportions than an adult, and a man may have different dimensions and proportions than a woman. Further, people of different backgrounds and ethnicities can have different proportions and dimensions, and so the head support including a chin rest and/or a forehead support would need to be adjusted.

The mechanical subsystem may be configured to position the head support and/or the at least two examination devices relatively with at least three degrees of freedom, preferably four degrees of freedom, further preferably five degrees of freedom and even further preferably with six degrees of freedom. Thus one or more of the head support and the at least two examination devices may be raised and lowered (moved in the Y-axis direction), moved from side to side (moved in the X-axis direction), moved forwards and backwards (moved in the Z-axis direction), and/or rotated about any one or more of the X, Y and Z axes. Such adjustment means may take the form of one or more toothed racks and pinions driven by a motor, a hydraulic device, a pneumatic device or another device. For this purpose, the X, Y and Z axes should be understood to be three orthogonal axes, which may not necessarily correspond with the X, Y and Z directions referred to earlier with respect to the reference point and/or surface.

The examination area may comprise a security panel for preventing a subject from moving too close to the examination machine or devices and moving parts. This panel may be provided with an aperture or opening to allow placement of just the portion of the subject to be examined in position on the support member, i.e. the portion of the head on the head support. The panel position can be adjusted so that the opening is at the correct height and/or lateral location for the particular subject, based on anthropometric data gathered or received by the sensor. Thus the panel may slide up and down or laterally (left and right), and/or may comprise a number of openings which may be selectively opened and closed to enlarge or reduce the size of the aperture.

The mechanical subsystem, may adjust the position and/or dimensions of any piece of furniture present in the examination area, for example may also include, raising and lowering and/or otherwise changing the position and orientation of a chair or seat on which the subject is seated, and/or may include a moving portion of the floor to allow for a variety of subject heights when standing or when seated such as in wheelchairs. Additionally, a table or surface on which a subject may lean during the course of the examination may be raised or lowered by the adjustment means, based on the feature data received.

The mechanical subsystem responds to output instructions provided by the control unit. The control unit as used herein may refer to a device, module, or subsystem having capability to receive data, and output the same or corresponding data to other electronics, for example a computer processor or controller. The control unit may comprise a processing unit for receiving input information and/or for providing output information. The control unit may be physically or remotely (e.g. wirelessly) connected to the system. The control unit may be connectable to a tangible medium such as a computer comprising computer readable instructions and further may comprise a control interface (e.g. panel) which as detailed herein may be used to insert/receive inputs and/or to present/provide output, optionally in accordance with pre-determined algorithms.

The control unit may receive any one or more of the following: feature data being representative of the at least one feature and examined data being representative of the eye parameters examined and as noted herein, in turn output instructions to the mechanical subsystem regarding the positioning of the head support and/or the at least two examination devices in the operative positions.

In some embodiments, the feature data may comprise data extracted from a photograph or image of the subject taken before the subject arrives at the subject examination area, for example from the subject's mobile communication device; data used during a previous examination of the same subject; data stored in a database related to the subject, e.g. data entered by the subject when booking an examination appointment; data based on statistics appropriate to one or more of the gender, age, height, weight and ethnicity of the subject; and/or data captured when the subject arrives at the examination area using a sensor.

The mechanical subsystem may be configured to lock the head support in position once the correct adjustments have been made to prevent accidental readjustment due to the weight of a portion of a subject, e.g. when leaning on the head support.

The examination system may comprise at least one of a biometric, a dental, a dermatological and/or an ear, nose and throat, ENT, examination device, and the at least one head support may comprise at least one of a forehead support and a chin support.

It is envisaged that the system and method of the present disclosure are alternative or additionally suitable for use with any other examination devices, such as biometric, a dental, a dermatological and/or an ear, nose and throat, ENT, examination device

Each examination area may comprise two head supports, for example, one each of a chin and forehead support. Chin and forehead supports provide a stable and supportive arrangement for a head of a subject to prevent accidental movement while the eyes, mouth or other facial tissue are being examined.

In some examples, the mechanical subsystem can be configured to adjust a height of, and/or a distance between, a forehead support and a chin support.

The at least two ophthalmological examination devices can be configured to be positioned operatively simultaneously or sequentially. The at least two ophthalmological examination devices can be configured to be operated simultaneously or sequentially. In this manner, examination data can be gathered more efficiently, either in terms of time savings and/or in terms of the subject not needing to change location between examination devices. In some examples, there tray be more than two types of examination devices, and a controller can determine which should be brought to the subject and in what order.

In some embodiments, the system can comprise a plurality of said head supports, such that the system is capable of examining multiple subjects simultaneously, each subject being located at a different one of said plurality of said head supports. Each of the head supports may be located in a separate one of a plurality of subject examination areas. Such areas may be separated by fixed or removable walls to afford privacy and set space to a subject. For the system comprising a plurality of said head supports, the control unit being configured to position the at least two ophthalmological examination devices and/or the head support in a plurality of operative positions, one for each pair of ophthalmological examination device and said head support

According to a second aspect of the present disclosure, there is provided an examination station comprising at least two ophthalmological examination systems according to any of the embodiments of the first aspect. The examination station is capable of examining at least two subjects, each subject being examined by a different ophthalmological examination system. Each of the head supports for supporting a portion of a subject's head may be located in a separate one of a plurality of subject examination areas. Such areas may be separated by fixed or removable walls to afford privacy and set space to a subject. The at least two subjects may be examined simultaneously.

The system of the first aspect, or each system or the station, of the second aspect may comprise a coordination or system controller, configured to coordinate allocation and guidance of ophthalmological examination devices to and from operative positions relative to the at least two subjects. With this arrangement, multiple subjects may be assessed or examined simultaneously or in quick succession, providing time savings and increasing efficiency. The coordination or the system controller may be considered as part of the control unit.

The coordination or system controller may be operatively connected to a guiding mechanism, controlled by the system controller, for guiding a desired. ophthalmological examination machine, or other examination device, to a selected one of the subject examination areas.

Examination devices may comprise at least one of a biometric, a dental, a dermatological, an ear, nose and throat, ENT, in addition to the at least two ophthalmological examination devices. Ophthalmological machines may include machines such as those for carrying out Auto Refraction, Topography, Tonometry, Pachymetry, Retinal Imaging, Posterior Optical Coherence Tomography (OCT), Anterior OCT, Biometry, Specular Microscopy or Subjective Refractions, etc.

The system controller may comprise a processing unit for receiving input information and/or for providing output information. The system controller may he physically or remotely (e.g. wirelessly) connected to the system or station. The system controller may be connectable to a tangible medium such as a computer comprising computer readable instructions and further may comprise a control interface (e.g. panel) which as detailed herein may be used. to insert/receive inputs and/or to present/provide output, optionally in accordance with pre-determined algorithms.

The guiding mechanism may be capable of at least one of raising and lowering an examination machine (along a Z-axis), moving an examination machine closer towards and away from the selected one of the subject examination areas (along a Y-axis) and moving an examination machine laterally with respect to the selected one of the subject examination areas (along an X-axis). The guiding mechanism may additionally be capable of rotating (i.e. changing an orientation of) an examination machine relative to the selected one of the subject examination areas, e.g. rotation about at least one of the X, Y and Z axes. The guiding mechanism may be in the form of one or more racks with motors and pinions, to allow the guiding mechanism to guide the desired one of the at least one examination machine to a selected one of the subject examination areas. The guiding mechanism may form part of the mechanical subsystem and aid alignment of the examination machine with the subject in the subject examination area. For example, the guiding mechanism may raise, lower, change a distance between and/or laterally align the at least two ophthalmological examination devices with one or both eyes of the subject, while in a dental examination system, aligning the examination device with a part of the mouth or jaw.

The system or station of the first and second aspects can comprise a storage area for ophthalmological examination devices not in use.

The storage area may be a central area, and the plurality of subject examination areas, or head supports, may be arranged around the central area. The central area may be circular, rectangular, square or any other shape in plan. By central area, it is envisaged an area which is central in its location relative to the head supports, or alternatively an area providing shared access to examination devices which may not necessarily be surrounded in whole or in part by the head supports.

The plurality of subject examination areas, or head supports, may be arranged in at least one linear formation. The arrangement may be vertical or horizontal. The guiding mechanism may comprise at least one of: a linear translation mechanism, for example, such as a conveying means like a moving belt, configured, in use, to align a desired examination machine, with a selected one of the at least one subject examination areas or head supports.

Alternatively or additionally, the plurality of subject examination areas may be arranged in an annular or partially annular, i.e. arcuate, formation. In this arrangement, the system or station may comprise a rotatable platform comprising at least one tier of slots as a storage area for storing the at least one examination machine when not in use. The mechanical subsystem may comprise the platform, whereby rotation of the platform is configured, in use, to align a desired examination machine, or empty slot on the platform, radially with a selected subject examination area or head support. The mechanical subsystem may comprise a vertical displacement mechanism for vertically displacing the desired examination machine between the platform and a selected subject examination area and head support, and an alignment mechanism for aligning the desired examination machine with a subject to be examined at a selected subject examination area or head support.

The use of an annular or arcuate formation of subject examination areas at least partially surrounding a central rotatable platform allows a compact arrangement whereby the plurality of examination machines may be compactly stored radially centrally, while the relatively radially outwardly located subject examination areas are larger for a subject. Additionally, or alternatively, more subject examination areas may be able to surround a single rotatable platform. In this case, one or more of the rotatable platform and. the subject examination areas may comprise more than one tier, for example, two tiers, in order to be able to store enough examination machines for the number of subject examination areas, or to provide enough subject examination areas for subjects. For example, each tier of a two-tier rotatable platform may comprise four slots, allowing storage of eight examination machines. The rotatable platform may be surrounded by eight subject examination areas, or if two-tiered, sixteen subject examination areas. Other possible arrangements are envisaged, e.g. four subject examination areas surrounding a two-tiered platform comprising at least two slots on each level, or sixteen-people subject examination areas surrounding a two-tiered platform comprising at least eight slots an each level, etc.

In some embodiments, the subject examination areas can be arranged in at least one linear formation and the system may comprise a rotatable platform comprising at least one tier of slots for storing the at least one examination machine when not in use, conjunction with a linear conveying means such as a moving belt to convey a select examination machine from the rotatable platform to a select subject examination area, and back again, or none subject examination area to another subject examination area if the examination machine is hooked for sequential use by two or more different subjects. Such a rotatable platform may be a compact means of storing e machines, while the at least one linear formation can utilize a space which would not be large enough or suitably shaped to allow an annular formation of subject examination areas.

Since the platform can be rotated, a desired examination machine can be radially aligned, to face a selected subject examination area or a particular conveying means. The rotatable platform may comprise one or more ball bearings and races, motors and the like. Such mechanisms allow both support and translation of the heavy load of the examination machines.

The system controller of the system or station may comprise a placement controller for controlling removal and replacement locations of examination machines. This may be to ensure that the examination machines are put back in the same slots from which they were originally removed, or may be to ensure that the rotatable platform is balanced by storing the examination machines in the same slots or different slots from which they were originally removed, i.e. so that there is not an imbalance between one side of the platform being fully loaded on one or more tiers and the other side of the platform being empty of examination machines. Alternatively, or additionally, the examination machines may be replaced into appropriate slots which optimize the transfer of the examination machines to subsequent users, i.e. to reduce waiting time.

In some embodiments, the rotatable platform may be configured to comprise as many, or more, examination machines than subject examination areas. In such an arrangement, there may be several of one type of examination machine so that a subject will not need to wait too long for a particular examination when multiple patients are being simultaneously examined.

The system controller may take account of what examination machines are required for each subject examination area and optimize the order of examinations and use of the examination machines in each subject examination area, to minimize waiting time in each subject examination area.

The vertical displacement mechanism allows vertical displacement of the desired one of the plurality of examination machines between the platform and the subject examination area. The rotatable platform may be higher or lower than the selected subject examination area, so that the desired examination machine may need to be raised or lowered between the rotatable platform and the subject examination area, i.e. along a Z-axis. The use of vertical space is efficient for storage of the examination machines while minimizing the total area taken up by the system. There may be provided a separate vertical displacement mechanism for each subject area, or there may be shared one or more vertical displacement mechanisms capable of moving around the system to vertically displace desired examination machines.

The vertical displacement mechanism may comprise one or mare racks, pinions and motors to raise and lower a holding area. The vertical displacement mechanism may he provided with a gripping mechanism to safely grasp a machine, and a shifting mechanism to move the machine between the holding area and the empty slot on the platform.

The alignment mechanism allows alignment of the desired one of the plurality of examination machines with a subject to be examined at the selected one of the subject examination areas. This alignment may be in two orthogonal directions, far example a radial. direction (along a Y-axis), towards and away from the subject examination area and a lateral direction (along an X-axis), i.e. side to side relative to a subject in the subject examination area. Additionally, or alternatively, the alignment mechanism may allow rotation of the examination machine about any of the X, Y and Z axes.

The alignment mechanism may comprise one or more of a moving belt, rollers and lever arms. Alternatively, or additionally, the vertical displacement mechanism and the alignment mechanism may be at least partially combined. For example, the shifting mechanism of the vertical displacement mechanism may carry out at the least the alignment along the Y-axis direction and may also carry out the alignment along the X-axis direction. For example, the shifting mechanism may comprise telescoping arms, or a platform on rollers, capable of vertical movement through a raising and lowering mechanism together with a lateral displacement mechanism.

The mechanical subsystem may be controlled by the system controller (optionally the control unit).

In any of the first and second aspects, each head support may comprise a contact surface arranged, in use, to contact at least a part of a subject. The examination system may comprise a cleaning mechanism for providing a clean contact surface; a presence sensor for sensing the presence of a subject to be examined; and a controller for automatically activating the cleaning mechanism in response to a signal from the presence sensor.

By “automatic”, what is meant is without the need for human or operator intervention, or any external interference, in order to activate, or cause the controller to activate, the cleaning mechanism. Specifically, the self-operation is done in response to the signal that is provided by the presence sensor as an output and received by the cleaning mechanism as an input. Upon receipt of the signal, the cleaning mechanism is automatically operated to provide a clean contact surface. This arrangement is thus fully automated, i.e. automatically self-operated to provide a clean contact surface for contacting a subject to be examined.

The term “sensor” in “presence sensor” as used herein refers in its broadest definition to a device, module, or subsystem having capability to detect an event or a change in a predefined surroundings, and send the information to other electronics, for example a computer processor or controller. The controller may comprise a processing unit for receiving input information and/or for providing output information. The controller may be physically or remotely (e.g. wirelessly) connected to the apparatus. The controller may be connectable to a tangible medium such as a computer comprising computer readable instructions and further may comprise a control interface (e.g. panel) which as detailed herein may be used to insert/receive inputs and/or to present/provide output, optionally in accordance with pre-determined algorithms.

The contact surface may be a surface of the head support. Since the contact surface is part of the head support which will be cleaned, this arrangement is environmentally friendly.

The head support may comprise at least one roller. A roller can be rotated so that only a portion of the roller faces and contacts the subject to be examined. In this way the portion of the roller facing away from the subject to be examined can be cleaned while the portion facing towards the subject to be examined is in use. Subsequently, the roller can be rotated and the used side can be cleaned while the cleaned side can be used immediately. The roller may comprise a cleanable contact surface with a cushioned under-layer far comfort of the subject to be examined while being supported by the head support as well as ensuring a sanitary surface.

The contact surface may alternatively be a surface of a thin film material arranged to be supported by the head support. The thin film material may be flexible and serves to cover a more permanent, stiff and supportive head support. Thus a large contact surface between a subject to be examined and a user may be provided. Additionally, the shape of the contact surface can be adapted to more readily conform to the part of the subject to be examined, since the underlying head support can be correspondingly shaped and the thin, flexible film can conform to the shape of the head support. The thin film material may be adjacent to the head support, or may be spaced from the head support until a subject to be examined contacts and pushes against the thin film material.

The thin film material may comprise a continuous rotating belt. Thus the thin film material may be rotated around, with a portion not in contact with a subject to be examined being cleaned while another portion is in use. For example, the belt may be at least twice as long as the contact surface.

The cleaning mechanism may comprise at least one of a self-cleaning coating and a self-cleaning texture on the contact surface. Such self-cleaning coatings may be chemical coatings, while the self-cleaning textures maybe visible surface textures or microscopic surface textures, any or all of which may render the surface super-hydrophobic, super-hydrophilic, anti-microbial or having other self-cleaning properties which prevent adherence of bacteria and other dirt or germs, or which facilitate the ease of removal of these contaminants.

The cleaning mechanism. may comprise at least one of rollers, brushes, adhesive surfaces and other friction or adhesion members, arranged, in use, to clean the contact surface. The rollers and brushes may comprise a natural or synthetic material, and may have flexible bristles. The brushes may be straight brushes, cylindrical brushes or roller brushes. The brushes may have fixed axes of rotation, or the axes of rotation may be translated or rotated allowing thorough cleaning at more angles to take place. The cleaning mechanism may be capable of gripping or holding onto dirt particles, for examples, comprising high-friction materials such as silicone rubber, or comprising adhesive surfaces.

The cleaning mechanism may alternatively or additionally comprise a fluid suction action for drawing in a fluid, such as air or a liquid, together with contaminants on the contact surface.

The cleaning mechanism may comprise a fluid for cleaning the contact surface. Such a fluid may be applied to the contact surface and may subsequently evaporate, for example if it comprises a volatile solvent, may be sucked away from the contact surface, or may be dried from the contact surface using an absorbent device, blown air or heating. The fluid may comprise one or more of a gas and. a liquid. The fluid. may be applied using one or more of a brush, nozzle, a fine spray, a sponge or a permanent or disposable wipe and may similarly be sucked away by a nozzle or absorbed by a sponge. Any wipe, sponge or brush or other component of the cleaning mechanism which comes into contact with the contact surface may be permanent or disposable.

The fluid may comprise an antiseptic component, and/or a disinfectant. For example, the antiseptic may comprise an alcohol such as ethanol or 2-propanol/isopropanol. Use of an antiseptic component allows re-use of any components of the cleaning mechanism which come into contact with the contact surface since any germs or bacteria transferred to these wilt be killed by the action of the antiseptic component. Furthermore, use of an antiseptic component ensures that any bacteria which may not be removed by friction or other means of cleaning will be killed in place, and will not cause any harm to a subsequent subject to be examined.

The fluid, whether or not it contains an antiseptic component, may comprise various aromas, scents or other chemicals which freshens and gives an appealing and clean odor to the contact surface. In this manner, a contact surface can be used on multiple occasions without accumulating an undesirable odor, and can even be made appealing to a subject to be examined, in particular if it is a portion of the body such as the face (which contains the sensory smell receptors) which will need to be supported by the head support and contacted by the contact surface.

The cleaning mechanism may be at least one of a non-toxic, biodegradable and disposable material.

The cleaning mechanism may comprise a light source.

The thin film material may comprise a strip of paper, gauze or other surface. The cleaning mechanism may comprise two rollers onto which the thin film material is wound and between which the thin film material is supported. In use, the cleaning mechanism may be arranged to wind a used section of the thin film material onto one of the rollers, and to dispense an unused section of the thin film material from the other of the rollers.

Such a thin-film material may be absorbent and the used portion of this can be disposed of after a single subject has used it. Since the material is a thin film material, and will be disposed of eventually, more material can be compactly stored on a roller, ensuring that a roll will last longer before being changed. The thickness of the material should be thin enough to store compactly and prevent wastage of material, while thick enough to prevent accidental tearing while in use, for example if a subject perspires onto the strip, or when being rolled. The used section of the material can be wound onto a waste-side roller to ensure compact disposal of the used material. Alternatively however, the used material can be grasped between two waste-side rollers and rolled into a waste bin. In either case, the section of the material in use is held in tension between the rollers, preventing accidental back-movement of used sections of the material and avoiding unnecessary unravelling of fresh material. The rollers may be sprung-wound to effect a tight tension. The waste-side roller may comprise a pawl and ratchet mechanism, or other locking mechanism, for preventing rotation of the roller in an undesirable orientation which would bring used material back from the waste. Similarly, the other roller, i.e. supply-side roller, may comprise a locking mechanism preventing unnecessary dispensing of unused material.

Alternatively, the thin film material may comprise a replaceable pad of paper, gauze or other material, which may be joined in one or more locations by means of an adhesive or a fastening member such as a pin or a staple, and the cleaning mechanism may comprise an adhesive member or arm capable of peeling or tearing off a top layer of the pad. The thin film material may be at least one of a non-toxic, biodegradable and disposable material, and in some embodiments, may be a natural fabric, a synthetic fabric, a paper or a polymeric material.

In accordance with some embodiments, in response to a signal, a cleaning mechanism can be automatically operated to provide a clean contact surface. The signal may correspond to a change in the sensed presence of a subject to be examined. For example, if a subject to be examined was previously detected as being present by the presence sensor, and subsequently detected by the presence sensor as being absent (e.g. lack of presence), a signal may be sent to the controller to cause the cleaning mechanism to be activated. This ensures that once a subject to be examined has finished being examined and moves away from the contact surface, a clean contact surface will he automatically provided in readiness for the next subject to be examined. In this case, the controller may activate the cleaning mechanism after receiving the signal for the duration of a first pre-determined time period. This ensures that accidental and unnecessary cleaning does not take place should the subject being examined take a short break or rest from examination. Alternatively, and/or additionally, if no subject to be examined was previously detected. by the presence sensor (e.g. lack of presence), and subsequently a subject to be examined is detected by the presence sensor as being present, a signal may be sent to the controller to cause the cleaning mechanism to be activated. This ensures that once a subject to be examined has arrived and moves towards the contact surface, a clean contact surface will be automatically provided in readiness for the subject to be examined. In this case, the controller may activate the cleaning mechanism immediately upon receiving the signal, as long as the lack of presence was for a duration of at least a second pre-determined time period. This ensures that the clean contact surface will he ready for the subject to be examined without excess unnecessary cleaning should the subject to be examined have been returning from a short break or rest from examination.

According to a third aspect of the present disclosure, there is provided a method for automatic ophthalmological examination, comprising: detecting at least one feature of a subject to be examined using at least one sensor in a measuring subsystem; receiving feature data from the measuring subsystem representative of the at least one feature; determining, based on the received feature data, operative positions of at least two ophthalmological examination devices, the operative positions being selected to permit the at least two ophthalmological examination devices to examine one or both eyes of the subject; positioning a head support configured to support at least one portion of a head of a subject to be examined and/or at least two ophthalmological examination devices in operative positions; examining the subject using the at least two ophthalmological examination devices; receiving examination data from the at least two ophthalmological examination devices representative of eye parameters examined thereby; and outputting the examination data, or data or one or more image representative thereof.

The head support can be configured to support a chin and/or a forehead.

The at least one feature can be a facial feature. The at least one facial feature can be selected from a group consisting of chin, forehead, eyes and nose. The at least one facial feature can be one or both eyes.

The feature data can correspond to at least one of the distances eye to eye, pupil to pupil, eye to nose, eye to forehead, eye to chin, chin to forehead, nose to forehead and chin to nose.

The method can comprise measuring the at least one feature with respect to at least one reference surface or a reference point. The at least one reference surface can he located on a portion of an apparatus/system present throughout examinations, such as a floor or a portion of the head support.

Alternatively, or additionally, the method can comprise measuring the at least one feature with respect to at least one reference point. The at least one reference point can be a point located on a portion of an apparatus present throughout examinations, such as a floor or a portion of the head support.

The method can comprise determining a position of a chin or a forehead of the subject relative to the at least one reference surface and/or point.

Positioning the at least two ophthalmological examination devices in the operative position can comprise aligning an optical axis of each of the at least two ophthalmological examination devices with an optical axis of an eye of a subject.

The method can comprise positioning the at least two ophthalmological examination devices operatively simultaneously or sequentially. Alternatively or additionally, the method can comprise operating the at least two ophthalmological examination devices simultaneously or sequentially.

The method can comprise examining at least two subjects simultaneously, each subject being located at a different one of a plurality of said head supports.

The method can comprise coordinating allocation of, and guiding, ophthalmological examination devices to and from operative positions relative to the at least two subjects.

The method can comprise storing any ophthalmological examination devices not being used.

### Embodiments

Some embodiments of this disclosure will now be described in the following numbered paragraph. The following description intends to add on the above general description and not limit it in any manner.
1. A system for automatic ophthalmological examination of a subject, comprising:

a head support configured to support at least one portion of a head of the subject,

a measuring subsystem comprising at least one sensor configured to detect at least one feature of the subject;

a mechanical subsystem configured for receiving at least two ophthalmological examination devices and for positioning the at least two ophthalmological examination devices and/or the head support in operative positions; and

a control unit configured for:

(i) receiving feature data from the measuring subsystem representative of the at least one feature and determining the operative positions based thereon, the operative positions being selected to permit the at least two ophthalmological examination devices to examine one or both eyes of the subject,

(ii) outputting instructions to said mechanical subsystem for said positioning of the head support and/or the at least two ophthalmological examination devices in said operative positions,

(iii) receiving examination data from the at least two ophthalmological examination devices representative of eye parameters examined thereby, and

(iv) outputting the examination data or one or more image representative thereof.
2. The system according to Embodiment No. 1, wherein the head support s configured to support a chin and/or a forehead.
3. The system according to Embodiment No. 1 or 2, wherein the at least one feature is a facial feature.
4. The system according to Embodiment No. 3, wherein the at least one facial feature is selected from a group consisting of chin, forehead, eyes and nose.
5. The system according to Embodiment No. 4, wherein the at least one facial feature is one or both eyes.
6. The system according to any one of Embodiments No. 1 to 5, wherein the feature data corresponds to at least one of the distances eye to eye, pupil to pupil, eye to nose, eye to forehead, eye to chin, chin to forehead, nose to forehead and chin to nose.
7. The system according to any one of Embodiments No. 1 to 6, wherein the at least one feature is measured with respect to at least one reference point or surface.
8. The system according to Embodiment No. 7, wherein the at least one reference point or surface is with the system, e.g. a bottom surface of the system or said support
9. The system according to any one of Embodiment No. 7 or 8, wherein the control system is configured to determine a position of a chin or a forehead of the subject relative to the at least one reference surface and/or point.
10. The system according to any one of Embodiments No. 1 to 9, wherein the at least two ophthalmological examination devices are each positioned in the operative positions based on alignment of an optical axis thereof with an optical axis of an eye of a subject.
11. The system according to any one of Embodiments No. 1 to 10, wherein the at least two ophthalmological examination devices are configured to be positioned in the operative position simultaneously or sequentially.
12. The system according to any one of Embodiments No. 1 to 11, comprising a plurality of said head supports, and the control system being configured to position the at least two ophthalmological examination devices and/or the head support in a plurality of operative positions, one for each pair of ophthalmological examination device and said head support.
13. An examination station comprising at least two ophthalmological examination systems according to any one of Embodiments No. 1 to 11, configured for simultaneous examination of at least two subjects.
14. The system according to Embodiment No. 12, or the station according to Embodiment No. 13, wherein the control unit comprises a coordination controller, configured to coordinate allocation and guidance of the ophthalmological examination devices to and from operative positions.
15. The system or the station according to Embodiment No. 14, comprising a storage area for the ophthalmological examination devices not in use.
16. A method for automatic ophthalmological examination of a subject, comprising:

detecting at least one feature of the subject using at least one sensor in a measuring subsystem;

receiving feature data from the measuring subsystem representative of the at least one feature;

determining, based on the received feature data, operative positions of at least two ophthalmological examination devices, the operative positions being selected to permit the at least two ophthalmological examination devices to examine one or both eyes of the subject;

positioning a head support configured to support at least one portion of a head ardor the at least two ophthalmological examination devices in operative positions,

examining at least one eye of the subject using the at least two ophthalmological examination devices;

receiving examination data from the at least two ophthalmological examination devices representative of eye parameters examined thereby, and

outputting the examination data or one or more image representative thereof.
17. The method according to Embodiment No. 16, wherein the head support is configured to support a chin and/or a forehead.
18. The method according to Embodiment No. 16 or 17, wherein the at least one feature is a facial feature.
19. The method according to Embodiment No. 18, wherein the at least one facial feature is selected from a group consisting of chin, forehead, eyes and nose.
20. The method according to Embodiment No. 19, at least one facial feature is one or both eyes.
21. The method according to any one of Embodiments No. 16 to 20, wherein the feature data corresponds to at least one of the distances eye to eye, pupil to pupil, eye to nose, eye to forehead, eye to chin, chin to forehead, nose to forehead and chin to nose.
22. The method according to any one of Embodiments No. 16 to 21, comprising measuring the at least one feature with respect to at least one reference point or surface.
23. The method according to Embodiment No. 22, wherein the at least one reference point or surface is with the system, e.g. a bottom surface of the system or said support
24. The method according to any one of Embodiment No. 22 or 23, comprising determining a position of a chin or a forehead of the subject relative to the at least one reference surface and/or point.
25. The method according to any one of Embodiments No. 16 to 24, wherein positioning the at least two ophthalmological examination devices in the operative position comprises aligning an optical axis of each of the at least two ophthalmological examination devices with an optical axis of an eye of a subject.
26. The method according to any one of Embodiments No. 16 to 25, further comprising positioning the at least two ophthalmological examination devices in the operative position simultaneously or sequentially.
27. The method according to any one of claims Embodiments No. 16 to 26, comprising examining at least two subjects simultaneously.
28. The method according to any one of Embodiments No. 16 to 27, comprising coordinating allocation of, and guiding, the ophthalmological examination devices to and from operative positions.
29. The method according to any one of Embodiments No. 16 to 28, comprising storing any of the ophthalmological examination devices not in use.
30. A system for automatic examination of a body part of a subject, comprising:

a support configured to support a the subject's body portion comprising said body part so as to fix said body part in a fixed examination position,

a measuring subsystem comprising at least one sensor configured to detect at least one feature of the subject;

a mechanical subsystem configured for receiving at least two examination devices and for positioning the at least two examination devices and/or said support in operative positions; and

a control unit configured for:

(i) receiving feature data from the measuring subsystem representative of the at least one feature and determining the operative positions based thereon, the operative positions being selected to permit the at the least two examination devices to examine said body part,

(ii) outputting instructions to said mechanical subsystem for said positioning of said support and/or the at least two examination devices in said operative positions,

(iii) receiving examination data from the at least two examination devices representative of parameters examined thereby, and

(iv) outputting the examination data or one or more image representative thereof,
31. The system of Embodiment No. 30, wherein said body part are teeth, eye or skin portion.
32. The system according to Embodiment No. 30 or 31, wherein the at least one feature is measured with respect to at least one reference point or surface.
33. The system according to Embodiment No. 32, wherein the at least one reference surface is with the system, e.g, a bottom surface of the system or said support.
34. The system according to any one of Embodiments No. 30 to 33, wherein the at least two examination devices are configured to be positioned in the operative position simultaneously or sequentially.
35. The system according to any one of Embodiments No. 30 to 34, comprising a plurality of said supports and the control system being configured to position the at least two examination devices and/or the support in a plurality of operative positions, one for each pair of device and said support.
36. An examination station comprising at least two examination systems according to any one of Embodiments No. 30 to 35, configured for simultaneous examination of at least two subjects.
37. The system according to Embodiment No. 35, or the station according to Embodiments No. 36, wherein the control unit comprises a coordination controller, configured to coordinate allocation and guidance of the examination devices to and from operative positions,
38. The system or the station according to Embodiment No. 37, further comprising a storage area for the examination devices not in use,
39. A method for automatic examination of a body part of a subject, comprising:

detecting at least one feature of the subject using at least one sensor in a measuring subsystem;

receiving feature data from the measuring subsystem representative of the at least one feature;

determining, based on the received feature data, operative positions of at least two examination devices, the operative positions being selected to permit the at least two examination devices to examine said body part;

positioning a support configured to support at least one body portion comprising said body part and/or the at least two examination devices in operative positions,

examining the at least one body part using the at least two examination devices;

receiving examination data from the at least two examination devices representative of parameters of the body part examined thereby, and

outputting the examination data or one ore more image representative thereof.
40. The method according to Embodiment No. 39, wherein the body part to be examined are teeth or skin.
41. The method according to any one of Embodiment No. 39 or 40, comprising measuring the at least one feature with respect to at least one reference point or surface.
42. The method according to Embodiment No. 41, wherein the at least one reference point or surface is with the system, e.g. a bottom surface of the system or said support.
43. The method according to any one of Embodiments No. 39 to 42, comprising determining a position of the body part relative to the at least one reference surface and/or point.
44. The method according to any one of Embodiments No. 39 to 43, further comprising positioning the at least two examination devices in the operative position simultaneously or sequentially.
45. The method according to any one of Embodiments No. 39 to 44, comprising examining at least two subjects simultaneously.
46. The method according to any one of Embodiments No. 39 to 45, comprising coordinating allocation of, and guiding the examination devices to and from operative positions.
47. The method according to any one of Embodiments No. 39 to 46, comprising storing any examination devices not in use.

### BRIEF DESCRIPTION OF THE DRAWINGS

In order to better understand the subject matter that is disclosed herein and to exemplify how it may be carried out in practice, embodiments will now be described, by way of non-limiting example only. with reference to the accompanying drawings, in which:

FIG. 1 shows a top rear perspective view of two apparatuses and. mechanism for it adjustment;

FIG. 2 shows a front view of the apparatuses and mechanism for heir adjustment as shown in FIG. 1;

FIG. 3 shows a top front perspective view of one of the apparatuses of is a forehead support;

FIG. 4 shows a top front perspective view of one of the apparatuses of FIG. 1 which is a chin support;

FIG. 5A shows a rear view of a toothed rack with sliders;

FIG. 5B shows a perspective view of a portion of the toothed rack of FIG. 5A;

FIG. 5C shows a perspective view of one of the sliders of FIG. 5A;

FIG. 6A shows a top perspective view of a motorized arrangement comprising a glider;

FIG. 6B shows a top perspective view of the glider of FIG. 6A;

FIGS. 7A-7D show top perspective views of various cleaning mechanisms for providing a clean contact surface for the forehead support of FIG. 3;

FIG. 8 shows a plan view of an ophthalmological examination system;

FIG. 9 shows a top perspective partially cut-away view of the ophthalmological examination system of FIG. 8;

FIGS. 10A and 10B show a side perspective view of a guiding mechanism for guiding an ophthalmological examination machine to a subject examination area;

FIGS. 11A and 11B show a plan view of an ophthalmological examination machine being moved in a Y-axis direction, towards and away from a subject examination area;

FIGS. 11C and 11D show a plan view of an ophthalmological examination machine being moved in an X-axis direction, towards and away from a subject examination area;

FIGS. 12A and 12B show a rear view of the two apparatuses of FIG. 1 in a subject examination area being adjusted in a Z-axis direction by the mechanism of FIGS. 1-6B;

FIG. 13 shows a top perspective view of ophthalmological examination system, showing for a particular subject examination area the X, Y and Z-axis directions;

FIG. 14 shows schematically a system for automatic ophthalmological examination; and

FIG. 15 shows schematically a method for automatic ophthalmological examination.

### DETAILED DESCRIPTION OF EMBODIMENTS

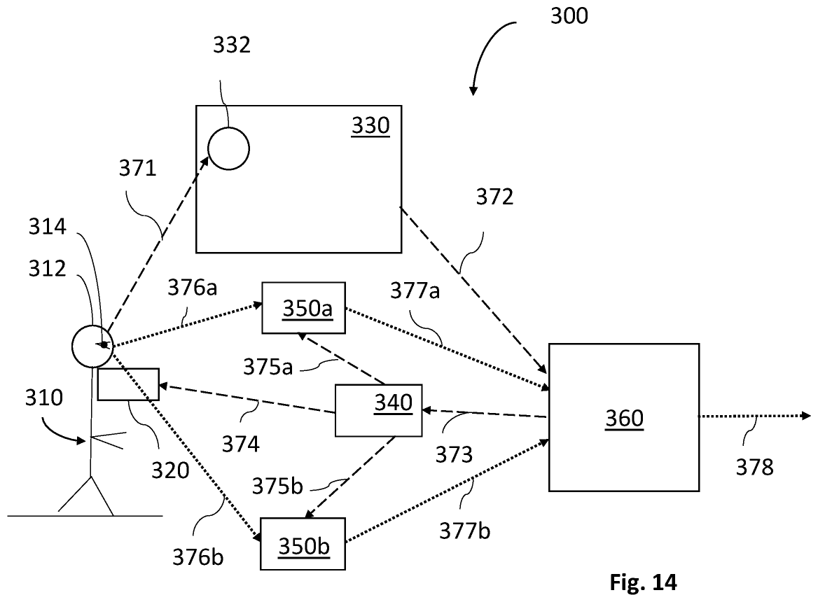

With reference to FIG. 14, there, is shown schematically a system for automatic ophthalmological examination. The system, generally designated 300, comprises a head support 320, a measuring subsystem 330, a mechanical subsystem 340 and a control unit 360.

The head support 320 is configured to support at least one portion of a head 312 of a subject 310 to be examined. The measuring subsystem 330 comprises at least one sensor 332 configured to detect at least one feature of the subject 310, as indicated schematically by dashed arrow 371.

The mechanical subsystem 340 is configured for receiving at least two ophthalmological examination devices 350a, 350b, and for positioning the at least two ophthalmological examination devices 350a, 350b and/or the head support 320 in operative positions, as indicated schematically by dashed arrows 375a, 375b and 374 respectively. While FIG. 14 shows two ophthalmological examination devices 350a and 350b, the mechanical subsystem 340 is configured for receiving more than two ophthalmological examination devices, for example three, four or more examination devices. If more than two ophthalmological examination devices are present, these would be denoted as 350a, 350b, 350c etc.

The control unit 360 is configured fir receiving feature data from the measuring subsystem 330, as indicated by dashed. arrow 372. The feature data is representative of the at least one feature detected by the at least one sensor 332. The control unit 360 is further configured for determining the operative positions based on the feature data. The operative positions are selected to permit the at least two ophthalmological examination devices 350a, 350b to examine one or both eyes 314 of the subject 310.

The control unit is also configured for outputting instructions to said mechanical subsystem 340 for said positioning of the head support 320 and/or the at least two ophthalmological examination devices 350a, 350b. in said operative positions, as indicated schematically by dashed arrow 373.

The control unit additionally is configured to receive examination data from the at least two ophthalmological examination devices 350a, 350b representative of eye parameters examined thereby, as indicated schematically by dotted arrows 376a, 376b, 377a, 377b. The control unit also is configured for outputting the examination data, or data or one or more image representative of the examination data, as indicated schematically by dotted arrow 378.

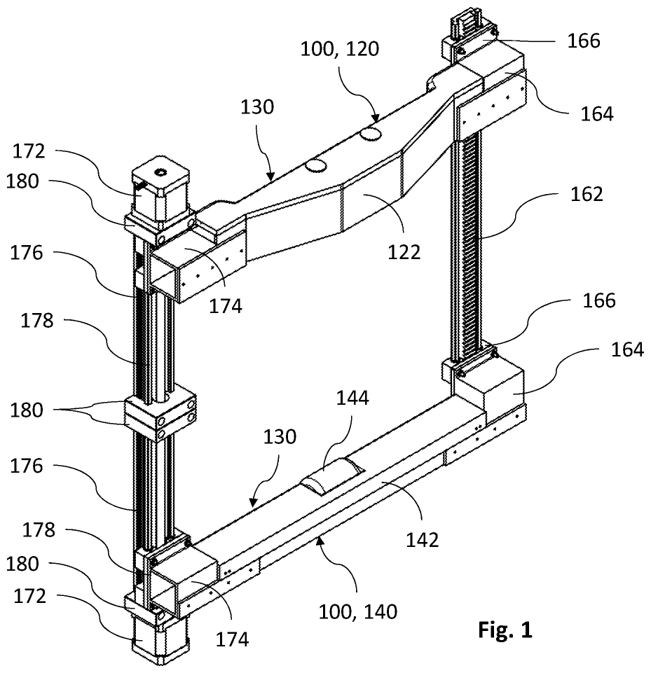

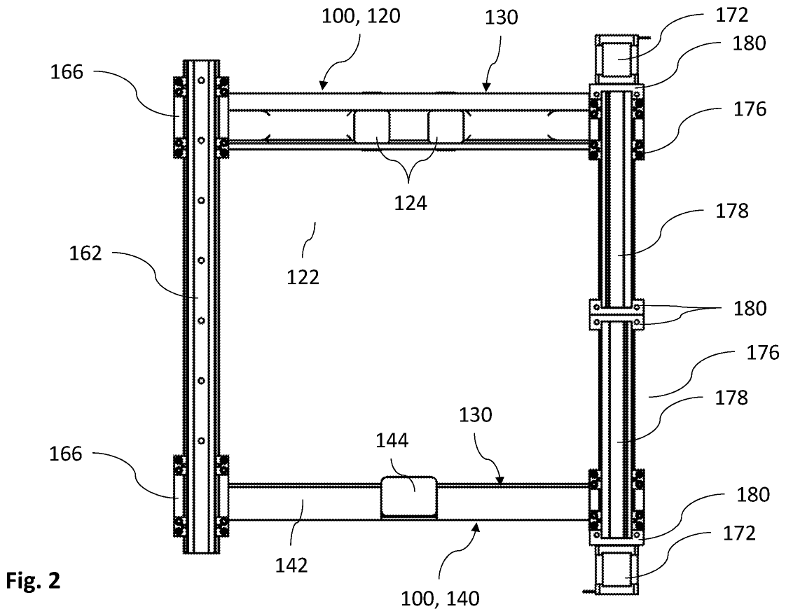

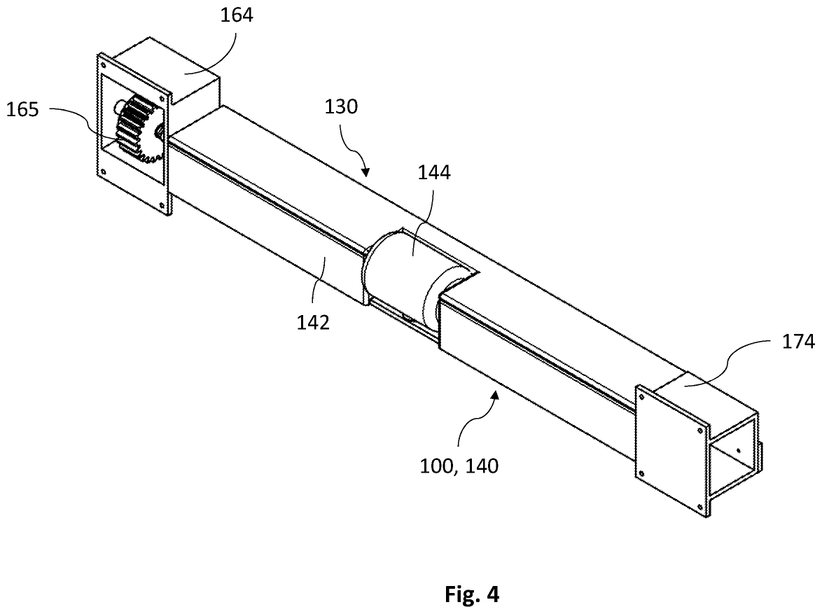

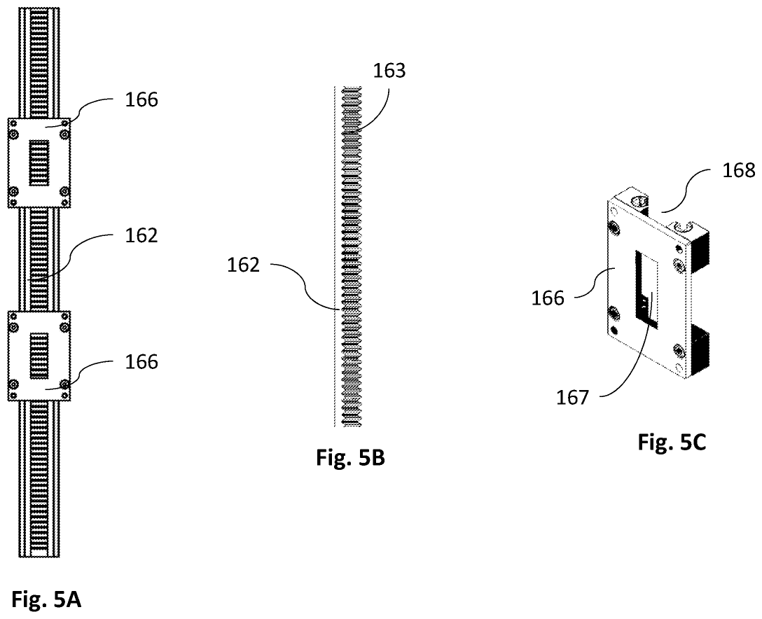

Further optional features of exemplary systems are described below with reference to FIGS. 1 to 13.

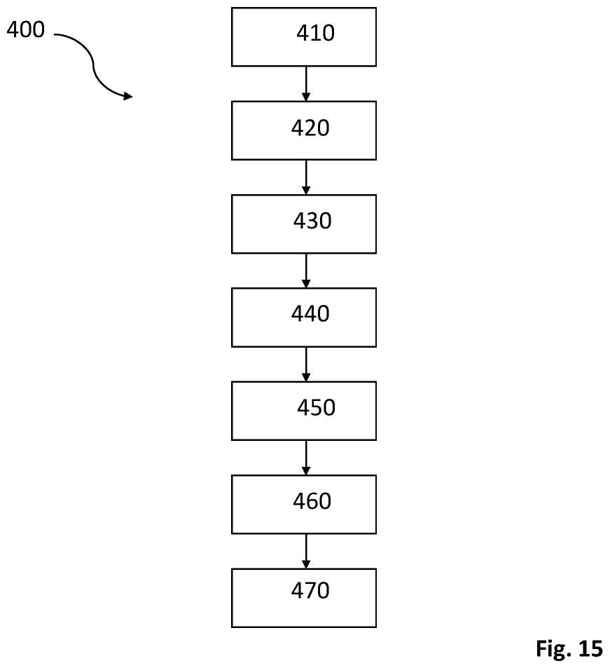

With reference to FIG. 15, there is shown schematically a method for automatic ophthalmological examination, generally designated 400. The method comprises, at step 410, detecting at least one feature of a subject to be examined using at least one sensor in a measuring subsystem. Such features may for example be a chin, nose, eyes,

At step 420, the method comprises receiving feature data from the measuring subsystem representative of the at least one feature. For example, feature data may include data representative of a distance between a subject's eyes, or a distance from a subject's chin to a reference surface such as a floor.

The method continues at step 430 to determine, based on the received feature data, operative positions of at least two ophthalmological examination devices, the operative positions being selected to permit the at least two ophthalmological examination devices to examine one or both eyes of the subject.

The method then continues at step 440, to position a head support configured to support at least one portion of a head of a subject to be examined and/or at least two ophthalmological examination devices in operative positions.

At step 450, the method comprises examining the subject using the at least two ophthalmological examination devices. Such examination may be simultaneous or successive (sequential), and may be used on both eyes or a single eye of a subject, for example.

At step 460, the method includes receiving examination data from the at least two ophthalmological examination devices representative of eye parameters examined thereby, and at step 470, the method includes outputting the examination data, or data or one or more image representative of the examination data.

Although not shown in the specific example of FIG. 15, the method may also include constantly monitoring and repositioning the ophthalmological examination devices and/or the head support during examination, i.e. throughout and/or between examinations, to maintain alignment of the subject with the examination machines and thus ensure obtaining accurate examination data.

As shown in the example of FIG. 1, a head support is provided in the form of two apparatuses 100, which in turn are in the form of a forehead support 120 and a chin support 140. The forehead support comprises a housing 122 from which two rollers 124 partially protrude, and the chin support 140 comprises a housing 142 from which a single roller 144 partially protrudes. Although in this example, the chin support 140 has a single roller 144 and the forehead support has two rollers, it is envisaged that the opposite may be the case, or each support may have one or more rollers. Each housing 122, 142 may comprise two or more portions which are arranged to be connected together to retain the roller or rollers 124, 144 in position.

Each of the forehead support 120 and a chin support 140 comprises at least one sensor 130 for sensing the presence of a subject to be examined. For example, the at least one sensor 130 may comprise one or more of a camera, infra-red sensor, a pressure sensor, temperature sensor, proximity sensor and light sensor. The at least one sensor 130 may be located at any one or more locations on the respective support, or adjacent to the support.

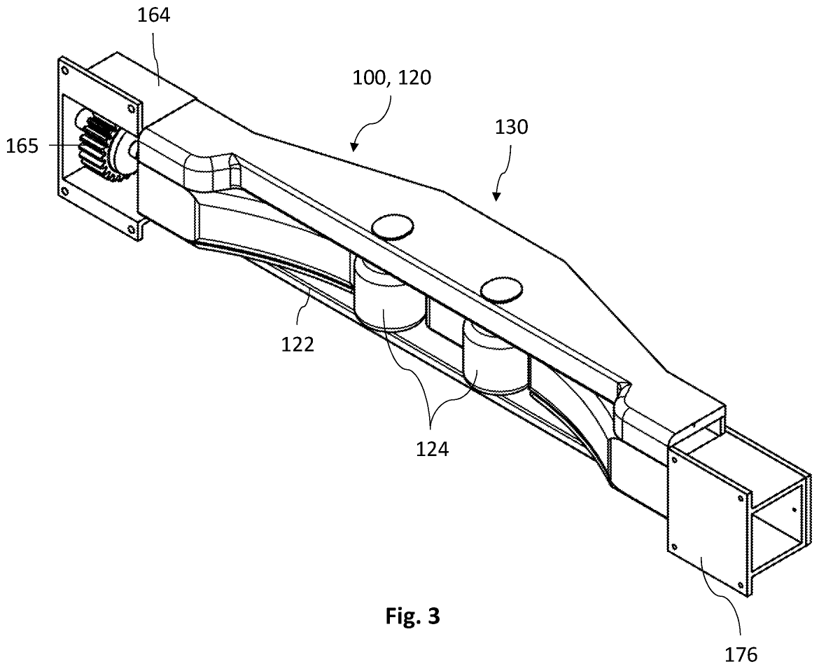

Each of the forehead support 120 and a chin support 140 comprises a first end portion 174 for operable connection to a motorized arrangement (described in more detail below), and a second end portion 164 for operable connection to a gear and rack sliding arrangement, which will now be described with reference to FIGS. 3, 4 and 5A-5C.

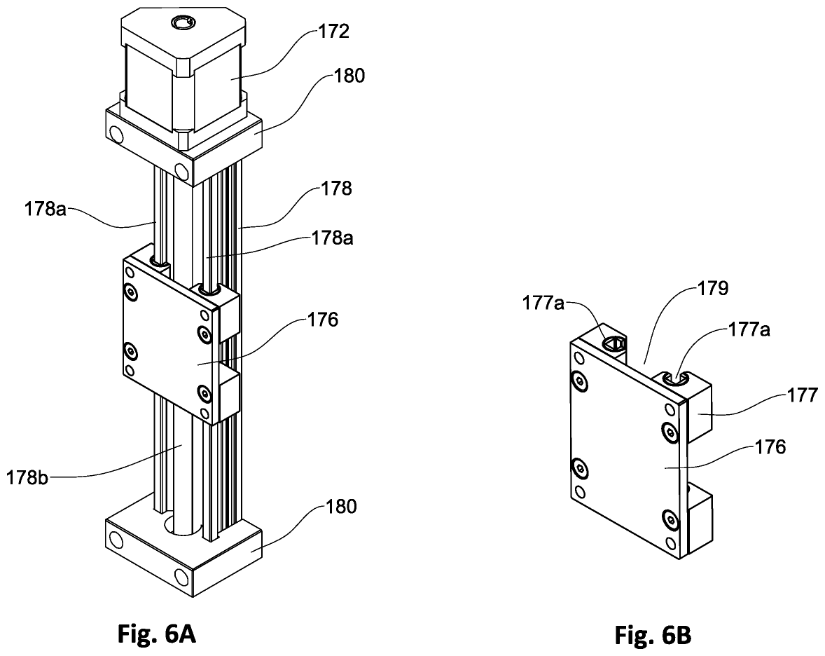

Each second end portion 164 houses a toothed gear 165 journaled for rotation about an axis, and protruding from the second end portion 164. Each gear is almost fully enclosed by a slider 166 connected to each end portion 164, with the exception that the slider 166 comprises an aperture 167 arranged to allow a portion of the gear 165 to protrude there through, for engagement with teeth 163 of a rack 162. The sliders 166 are closely fitted to the rack 162 by means of a recessed portion 168 in each slider 166, arranged to allow the rack 162 to pass there through. In operation, the sliders 166 can be translated along the length of the rack 162, with the toothed gears 165 rolling along and engaging with the teeth 163 of the rack, to allow the correct positioning of the chin support 140 and the forehead support 120. The translation of the sliders 166 along the length of the rack 162 is controlled by means of movement of the chin or forehead supports 140, 120 by a motorized arrangement, which will now be described with reference to FIGS. 6.A and 6B.

Each first end portion 174 is connected to a glider 176 comprising a recessed portion 179 arranged to allow a central shaft 178b to pass therethrough, and four protruding portions 177, each comprising a guide bore 177a arranged to allow a guide shaft 178a to pass there through. The protruding portions 177 are arranged such that the axes of the guide bores 177a of two protruding portions are aligned, i.e. coaxial, such that a single guide shaft 178a passes through two of the guide bores 177a. The guide shafts and guide bores may have a cross sectional shape other than circular, in order to prevent rotation of the guide shaft in the guide bores. For example, as shown in FIGS. 6A and 6B, the guide shafts 178a and guide bores 177a may comprise a square cross section. Other polygonal and non-axisymmetric cross sections are envisaged, for example rectangular triangular, pentagonal, hexagonal, oval, star-shaped, etc. Alternatively, the cross sections may be round, with a recessed key in one of the bores and the shafts being arranged to engage with a protruding key in the other of the bores and the shafts.

The central shaft 178b comprises a worm gear or other screw thread means, capable of engaging with a portion of the glider 176 and arranged to translate the glider 176 along a shaft path 178, which comprises the central and guide shafts 178b, 178a, and which is bounded at both ends by end-stops 180. At one end of the shaft path 178 is a motor 172 arranged to drive the central shaft 178b in order to translate the glider 176. Since the glider is attached to the first end portion 174 of the chin or forehead support 140, 120, translation of the glider causes translation of the chin or forehead support 140, 120, and thus also translation of the slider 166 at the second end 164 of the chin or forehead support 140, 120 along the toothed rack 163. In this manner, the height of, and spacing between, the chin and forehead supports 140, 120 can be adjusted.

In some embodiments, the chin and forehead supports 140, 120 can be adjusted roughly before a subject places his head against them, and can be finely adjusted to the correct alignment after the subject places his head against them. For at least this purpose, there may be a camera and/or other sensor on or near the chin and/or forehead supports, to ascertain where the subject to be examined is located. The exact positioning of the subject can be useful in enabling an examination machine to be correctly aligned and achieve the correct focus on the portion of the subject to be examined.

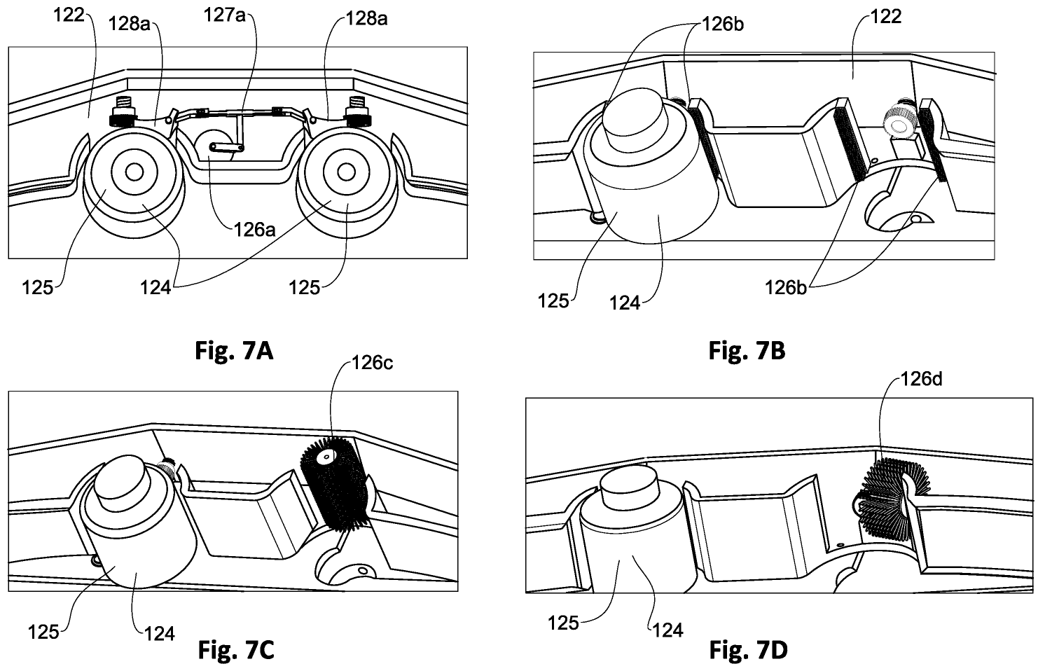

Various cleaning mechanisms with regard to the rollers 124 of the forehead support 120 will now be described, with reference to FIGS. 7A-D. The rollers 124 comprise a circumferential outer cylindrical surface, i.e. a contact surface 125, arranged to contact a part of a subject to be examined, i.e. a forehead. Since only a portion of the rollers 124 protrudes from the housing 122, the rollers 124 effectively comprise more than one contact surface 125, i.e. a contact surface 125 protruding from the housing 122 in contact with a subject, and at least another contact surface 125 contained within the housing 122 and arranged to be cleaned either by self-cleaning means such as surface coatings and textures, or by a cleaning mechanism which contacts the surface such as a brush, sponge or liquid, and/or by suction means which clean the contact surface.

In the example of FIG. 7A, the cleaning mechanism includes a rotating cleaning brush roller 126a moveable by a moving mechanism 127a to clean the rollers 125 in a car-wash style arrangement. In this arrangement, one or more actuation devices (not shown) are provided, which drive the moving mechanism 127a to bring at least one of the cleaning brush roller 126a or non-metallic cleaning scrapers 128a into frictional contact with the rollers 124 as they rotate. In the example of FIG. 7B, the cleaning mechanism includes four straight brushes 126b, each arranged on one side of a roller 124 to contact the roller 124 as it rotates past the brush 126b. Although four straight brushes 126b are shown, other numbers of brushes are envisaged. For example, at a minimum, only two straight brushes 126b are necessary, namely one straight brush 126b for each roller. In the example of FIG. 7C, the cleaning mechanism includes two cylindrical rotating brushes 126c (only one is shown), rotating about axes parallel to the axes of the rollers 124, each cleaning one of the rollers 124 as it rotates past the brush 126c. In the example of FIG. 7D, the cleaning mechanism includes two cylindrical rotating brushes 126d (only one is shown), rotating about axes perpendicular to the axes of the rollers 124, each cleaning one of the rollers 124 as it rotates past the brush 126d.

Although not shown in any of FIGS. 7A-7D, in any or all of the above examples, a fluid may be applied additionally to the rollers 124, and may optionally be removed by suction. The fluid may comprise an antiseptic component.

The cleaning mechanism cleans the contact surface 125 automatically, without the need for any human operator input, in response to at least one sensor 130 located in or on, or connected. operably to, each apparatus 100 (i.e. chin or forehead support 140, 120) sensing a change in the presence of a subject. The at least one sensor 130 may comprise one or more of a camera, infra-red sensor, a pressure sensor, temperature sensor, proximity sensor and light sensor. For example, the sensor may sense a new subject to be examined approaching, such as with a pressure sensor under the floor, a temperature sensor proximate to the apparatus, an infra-red beam being broken, a camera detecting movement, etc. At this point, in the examples of FIGS. 7A-7D, the rollers 124 on the forehead support 120 and roller 144 on the chin support 140 is rotated past the brushes, so as to provide a clean contact surface 125 of the rollers 124, 144 for contacting the forehead and chin of the subject to be examined respectively.

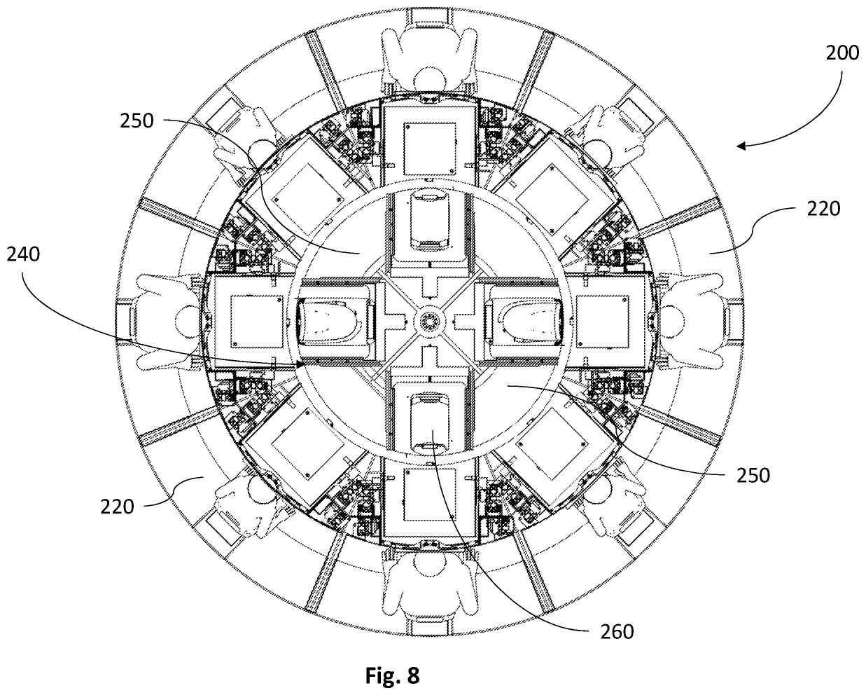

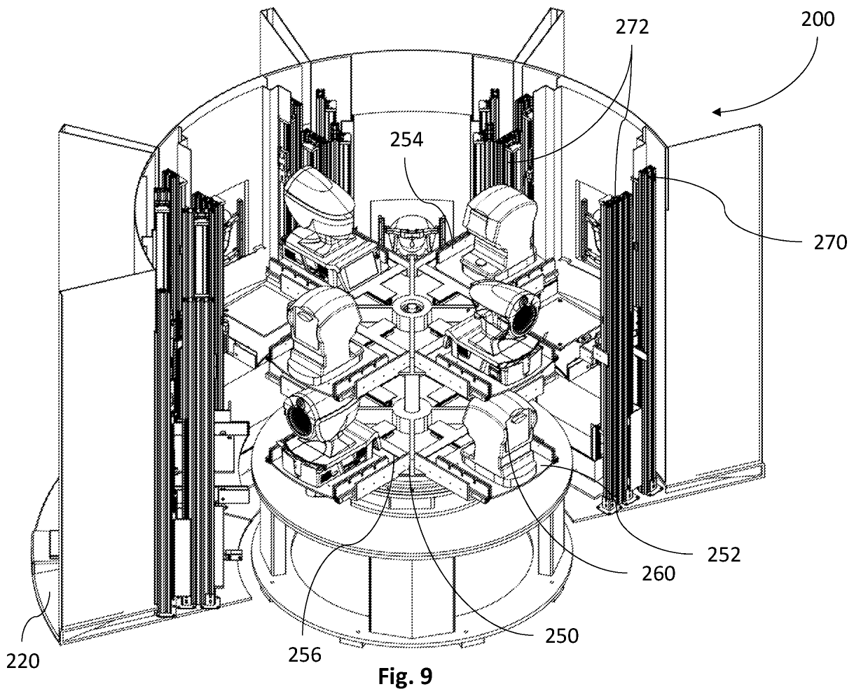

FIG. 8 depicts an exemplary system 200 comprising a plurality of subject examination areas 220 (in this example eight subject examination areas 220), arranged around a central area 240 comprising a plurality of ophthalmological machines 260, stored on a central rotating platform 250. Each examination area 220 comprises a head support. As shown more clearly in the cut-away section of FIG. 9, the central rotating platform 250 comprises a lower tier 252 and an upper tier 254, each comprising four slots 256 such that there is a capacity for storing up to eight ophthalmological examination machines 260 on the rotating platform 250.

The central rotating platform 250 is configured to be rotated until a desired ophthalmological examination machine 260 is radially aligned with a selected one of the subject examination areas 220. This is controlled by a central controller (not shown). Once this radial alignment is achieved, a guiding mechanism is used to move the desired ophthalmological examination machine 260 to the selected one of the subject examination areas 220. This may involve both vertical movement in a Z-axis direction, and horizontal movement, i.e. in a radial direction and in a tangential direction which is orthogonal to the radial direction. Thus, for each fixed subject examination areas 220, a radial direction between the subject examination area 220 and the center of the rotating platform 250 is defined as a Y-axis direction, and a tangential direction which is orthogonal to the Y-axis direction is defined as an X-axis direction.

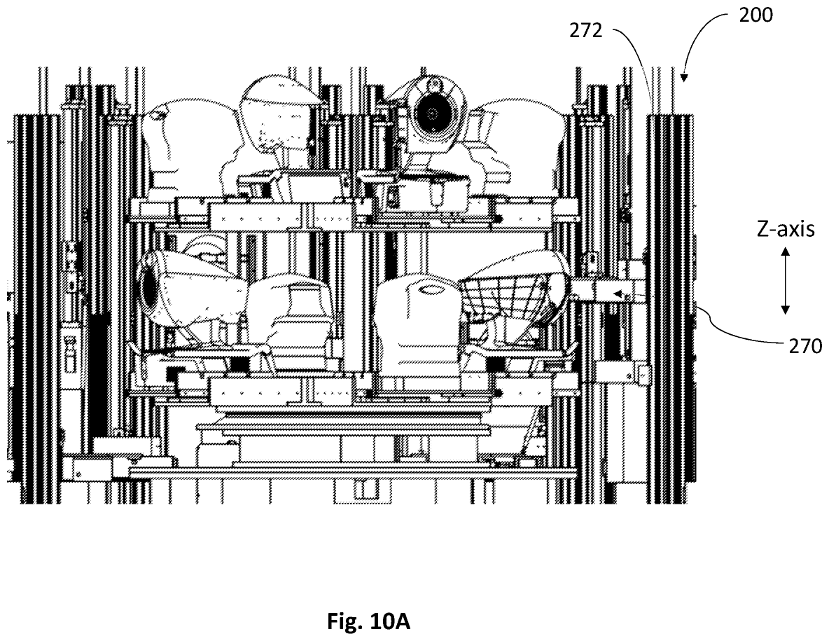

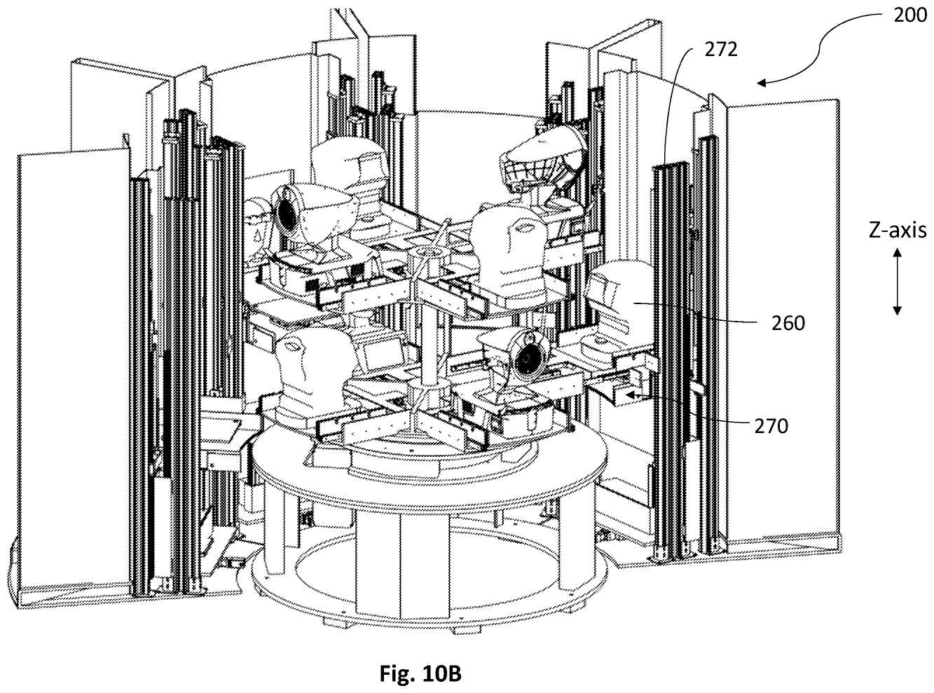

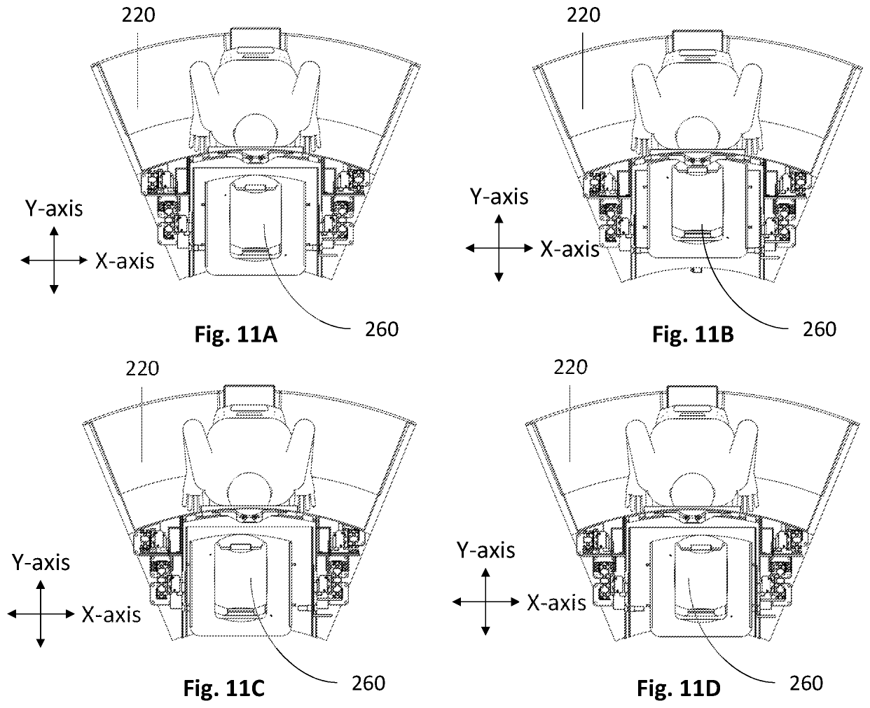

As shown in FIGS. 9, 10A and 10B, the exemplary guiding mechanism is separate for each subject examination area 220, and comprises a platform 270 which may be raised and lowered by the support of two side-posts 272, and which may be moved in the Y-axis direction by means of telescoping arms (see FIGS. 11A and 11B, showing the desired ophthalmological examination machine 260 being spaced further from and closer to the subject examination area 220 respectively), and may be adjusted in an X-axis direction (see FIGS. 11C and 11D) by means of rollers or the like, for example.

For ophthalmological examination, it is required. to stabilize the individual and specifically the eye position using a chair, a table and at least one support. Conventionally, an operator (eye healthcare professional) would manually adapt the ergonomic settings of the head support, the table top and the seat to the patient's morphology so that the eye or eyes of the subject would be in the correct position.

In the present example, within each subject examination area 220, various adjustment can be made to automatically accommodate the shape and form of each particular subject. The ophthalmological examination system 200 may comprise a data receiver (not shown) for receiving anthropometric data about a subject located in the subject examination area 220, and an ergonomic adjustment mechanism for automatically aligning at least one of the subject and a part of the subject examination area 220 in response to the received data, so that a portion of the subject to be examined is aligned with an examination location of the subject examination area 220 in response to the received data. By anthropometric data, what is meant is the proportions of the subject to be examined (for example, height when seated, size of head, position of eyes in the head, i.e. distance from chin and forehead, spacing between the eyes, etc.). The data receiver may receive any one or more of the following: data extracted from a photograph or image of the subject taken before the subject arrives at the subject examination area, for example from the subject's mobile communication device; data used during a previous examination of the same subject; data stored in a database related to the subject, e.g. data entered by the subject when booking an examination appointment; data based. on statistics appropriate to one or more of the gender, age, height, weight and ethnicity of the subject; or data captured when the subject arrives at the examination area using a sensor. The sensor may be any one or more sensors capable of sensing anthropometric data about a. subject to be examined. For example, the sensor may comprise one or more of a camera, infra-red sensor, pressure sensor, temperature sensor, proximity sensor and light sensor.

The ergonomic adjustment mechanism in this example comprises a chair or seat 222 which can move up and down, forwards and backwards (Y-axis direction), from side to side (X-axis direction) and/or rotate, and/or may include a moving portion of the floor of the subject examination area 220 which is suitable for the same adjustments, for use by subjects in wheelchairs. The ergonomic adjustment mechanism thus ensures that a portion of the subject to be examined—i.e. the eye or eyes—is correctly aligned with the examination area so that the placement is correct for examination.

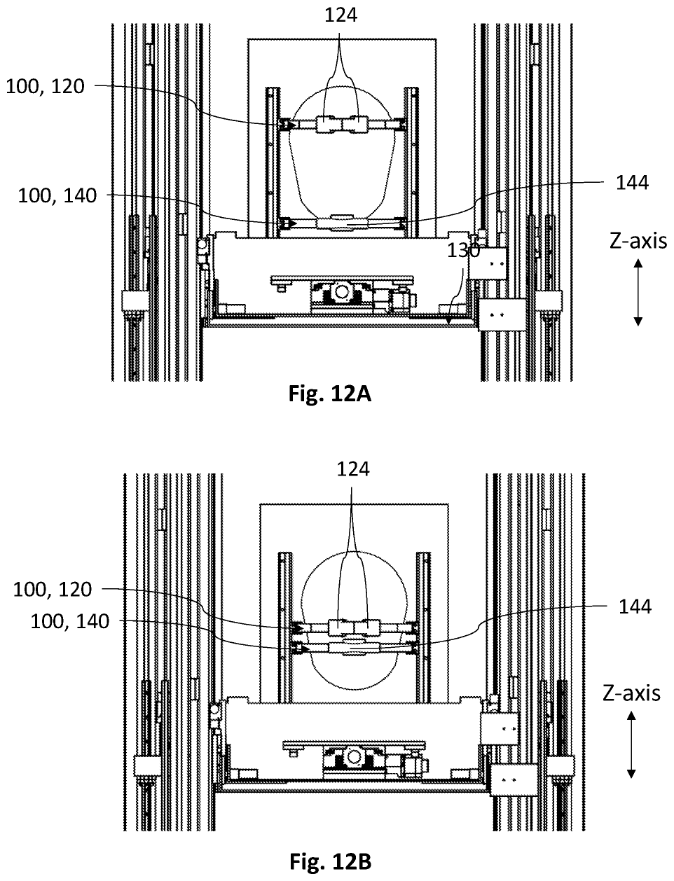

In this exemplary system, each of the subject examination areas comprises two apparatuses 100, namely a forehead support 120 and a chin support 140 as described above. As shown in FIGS. 12A and 12B, the ergonomic adjustment mechanism using the received data, e.g. relating to the size and height of the face of the subject to be examined, can adjust the height of, and spacing between, the chin support 140 and the forehead support 120 by moving these up and down, such as with the motorized system and rack, gear and sliders described above with reference to FIGS. 1-6B.

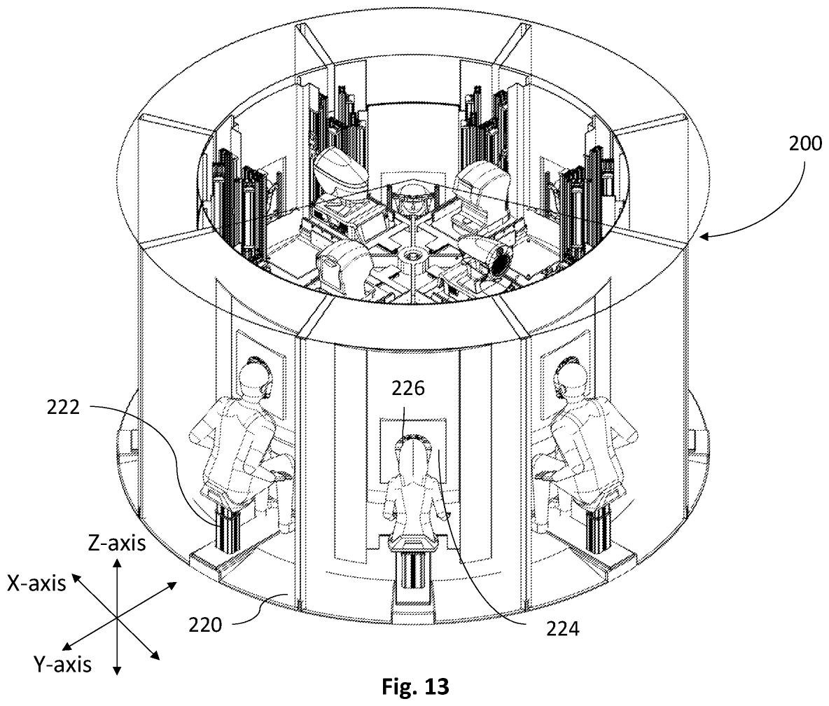

As shown in FIG. 13, the subject examination area may comprise a security panel 224 for preventing a subject moving too close to the examination machinery 260 and moving parts. This panel 224 may be provided with an aperture or opening 226 to allow placement of just the portion of the subject to be examined, in this case the face, in position on the apparatuses. The panel can be adjusted so that the opening is at the correct height and/or lateral location for the particular subject, based on anthropometric data gathered. Thus the panel may slide up and down or laterally, and/or may comprise a number of openings which may be selectively opened and closed to enlarge or reduce the size of the aperture 226.

Additionally, a table or surface on which a subject may lean during the course of the examination may be raised or lowered by the ergonomic adjustment mechanism, based on the patient's anthropometric data received by the data receiver.

Although not shown in the specific examples, it is envisaged that the sensor which detects features of a subject, in conjunction with the measurement subsystem, can define a safety zone in which features of the subject lie. This safety zone can constitute an invisible boundary which the controller communicates to the mechanical subsystem. In this way, the at least two examination devices can be positioned in such a manner which avoids collisions between the examination devices and the subject to be examined. The safety zone may additionally include the head support, such that collisions can also be avoided with the head support.

1. A system for automatic ophthalmological examination of one or both eyes of a subject, comprising:
a head support configured to support at least one portion of a head of the subject;
a measuring subsystem comprising at least one sensor configured to detect at least one feature of the subject;
a mechanical subsystem configured for receiving at least two ophthalmological examination devices and for positioning the at least two ophthalmological examination devices and/or the head support in operative positions; and
a control unit configured for:
(i) receiving feature data from the measuring subsystem representative of the at least one feature and determining the operative positions based thereon, the operative positions being selected to permit the at least two ophthalmological examination devices to examine one or both eyes of the subject,
(ii) outputting instructions to said mechanical subsystem for said positioning of the head support and/or the at least two ophthalmological examination devices in said operative positions,
(iii) receiving examination data from the at least two ophthalmological examination devices representative of eye parameters examined thereby, and
(iv) outputting the examination data or one or more image representative thereof.

2. The system according to claim 1, wherein the head support is configured to support a chin and/or a forehead.

3. The system according to claim 1, wherein the at least one feature is a facial feature.

4. The system according to claim 1, wherein the at least one feature is measured with respect to at least one reference point or surface.

5. The system according to claim 1, wherein the at least two ophthalmological examination devices are each positioned in the operative positions based on alignment of an optical axis thereof with an optical axis of an eye of a subject.

6. The system according to claim 1, comprising a plurality of said head supports, and the control unit being configured to position the at least two ophthalmological examination devices and/or the head support in a plurality of operative positions, one for each pair of ophthalmological examination device and said head support.

7. An examination station comprising at least two ophthalmological examination systems according to claim 1, configured for simultaneous examination of at least two subjects.

8. The system or the station according to claim 7, comprising a storage area for the ophthalmological examination devices not in use.

9. A method for automatic ophthalmological examination of a subject, comprising:
detecting at least one feature of the subject using at least one sensor in a measuring subsystem;
receiving feature data from the measuring subsystem representative of the at least one feature;
determining, based on the received feature data, operative positions of at least two ophthalmological examination devices, the operative positions being selected to permit the at least two ophthalmological examination devices to examine one or both eyes of the subject;
positioning a head support configured to support at least one portion of a head and/or the at least two ophthalmological examination devices in operative positions,
examining at least one eye of the subject using the at least two ophthalmological examination devices;
receiving examination data from the at least two ophthalmological examination devices representative of eye parameters examined thereby, and
outputting the examination data or one or more image representative thereof.

10. The method according to claim 9, wherein the head support is configured to support a chin and/or a forehead.

11. The method according to claim 9, wherein the at least one feature is a facial feature.

12. The method according to claim 9, wherein positioning the at least two ophthalmological examination devices in the operative position comprises aligning an optical axis of each of the at least two ophthalmological examination devices with an optical axis of an eye of a subject.

13. The method according to claim 9, comprising examining at least two subjects simultaneously.

14. The method according to claim 9, comprising coordinating allocation of, and guiding, the ophthalmological examination devices to and from operative positions.

15. The method according to claim 9, further comprising storing any of the ophthalmological examination devices not in use.

16. A system for automatic examination of a body part of a subject, comprising:
a support configured to support a subject's body portion comprising said body part so as to fix said body part in a fixed examination position,
a measuring subsystem comprising at least one sensor configured to detect at least one feature of the subject;
a mechanical subsystem configured for receiving at least two examination devices and for positioning the at least two examination devices and/or said support in operative positions; and
a control unit configured for:
(i) receiving feature data from the measuring subsystem representative of the at least one feature and determining the operative positions based thereon, the operative positions being selected to permit the at the least two examination devices to examine said body part,
(ii) outputting instructions to said mechanical subsystem for said positioning of said support and/or the at least two examination devices in said operative positions,
(iii) receiving examination data from the at least two examination devices representative of parameters examined thereby, and
(iv) outputting the examination data or one or more image representative thereof.

17. The system of claim 16, wherein said body part are teeth, eye or skin portion.

18.-19. (canceled)
